(19) United States
(12) Patent Application Publication
(10) Pub. No.: US 2013/0066257 A1
(43) Pub. Date: Mar. 14, 2013

(54) Design of Interventional Cutting Balloon with Enhanced API Delivery Capability

(71) Applicants: Martyn Folan, Loughrea (IE); John Clarke, Claregalway (IE); Aiden Flanagan, Kilcolgan (IE); Anthony Malone, Miltown (IE); Tim O'Connor, Claregalway (IE)

(72) Inventors: Martyn Folan, Loughrea (IE); John Clarke, Claregalway (IE); Aiden Flanagan, Kilcolgan (IE); Anthony Malone, Miltown (IE); Tim O'Connor, Claregalway (IE)

(73) Assignee: BOSTON SCIENTIFIC SCIMED, INC., Maple Grove, MN (US)

(21) Appl. No.: 13/608,237

(22) Filed: Sep. 10, 2012

Related U.S. Application Data
(60) Provisional application No. 61/533,592, filed on Sep. 12, 2011.

(51) Int. Cl. A61B 17/22; A61M 37/00

(52) U.S. Cl. 604/22; 606/159

(57) ABSTRACT

A medical balloon assembly including an inflatable balloon and at least one cutting element extending from the balloon is disclosed. The inflatable balloon includes an outer wall facing outward and an inner wall facing outward. The cutting element comprises a plurality of openings therein including a first opening and a second opening. The first opening is closed off by a portion of the outer wall. The cutting element further includes a passageway extending from the first opening to the second opening. The passageway is defined by the cutting element and the portion of the outer wall which closes off the passageway. The passageway may contain a therapeutic agent.

### FIELD

This disclosure relates to medical devices for use in dilating bodily vessels and delivering therapeutic agents thereto.

### BACKGROUND

Coronary and peripheral artery disease are conditions in which the bodily arteries and vessels (that provide oxygen and nutrients to the heart) become narrowed or blocked. It may be caused by atherosclerosis, an accumulation of fatty materials such as plaque on the inner linings of the arteries and associated vessels. These plaque deposits are most common in the peripheral blood vessels that feed the limbs of the human body and the coronary arteries that feed the heart. The resulting blockage restricts blood flow to the heart and peripheral locations.

Currently, balloon angioplasty or percutaneous transluminal coronary angioplasty (PTCA) procedures are being used in treating coronary and peripheral artery disease by reopening blocked or narrowed blood vessels. In balloon angioplasty, a balloon catheter is guided to the target lesion, where the physician or operator inflates a balloon to compress the plaque against the arterial wall and reestablish lumen patency. A cutting balloon may be used, where appropriate, to score or cut the plaque to facilitate an improved balloon dilation of the segmented lesion. The dilated lesion site may then be treated by introducing a drug eluting stent or balloon style device that provide a therapeutic agent-based therapy in the proximity of the previously scored lesion site. Delivery of therapeutic agents in this manner may give enhanced drug delivery via the prescored lesion locations to access the underlying vasculature tissue for improved therapeutic agent uptake. This technique typically involves a dual device treatment system in which the primary cutting balloon dilation device is followed by a secondary treatment procedure involving a drug eluting stent, balloon, or the like.

Therefore, a medical device and a treatment method within the patient's body is required to overcome the need of the dual device treatment procedure system for balloon angioplasty or PTCA by providing an improved combination device with benefits to cost and procedural times.

Without limiting the scope of the invention, a brief summary of some of the claimed embodiments of the invention is provided below. Additional details of the summarized embodiments of the invention and/or additional embodiments of the invention may be found in the detailed description of the invention.

A brief abstract of the technical disclosure in the specification is provided as well for the purposes of complying with 37 C.F.R. 1.72. The abstract is not intended to be used for interpreting the scope of the claims.

All US patents and applications and all other published documents mentioned anywhere in this application are incorporated herein by reference in their entirety.

### SUMMARY

In at least one embodiment, the invention is directed to a medical device comprising a balloon assembly where the balloon assembly comprises a balloon having an outer wall facing outward and an inner wall facing inward. The medical balloon assembly further includes a cutting element extending from the balloon and having a plurality of openings therein including a first opening and a second opening. The first opening is closed off by a portion of the outer wall. The cutting element further includes a passageway extending from the first opening to the second opening. The passageway is defined by the cutting element and the portion of the outer wall which closes off the passageway. The balloon may be disposed about a catheter.

In at least one embodiment, the invention is directed to a medical device comprising a balloon assembly and the balloon assembly comprises an inflatable balloon having an outer wall facing outward and an inner wall facing inward. The medical balloon assembly further includes a cutting element extending from the balloon and having a plurality of openings therein including a first opening and a second opening. The cutting element further includes a cavity extending from the first opening to the second opening. A portion of the outer wall extends across the first opening. The outer wall is constructed and arranged such that in an inflated state of the balloon, a portion of the outer wall extends into the cavity. The balloon may be disposed about a catheter.

In at least one embodiment, the invention is directed to a medical device comprising a balloon assembly where the balloon assembly comprises an inflatable balloon having a balloon wall. The medical balloon assembly further includes a cutting element extending from the balloon and having a plurality of openings therein including a first opening and a second opening. The cutting element further includes a cavity extending from the first opening to the second opening. The first opening is covered by a flexible piece of material which extends into the cavity upon inflation of the balloon. The balloon may be disposed about a catheter.

In at least one embodiment, the invention is directed to a medical device comprising a balloon assembly where the balloon assembly comprises a balloon having an outer wall facing outward and an inner wall facing inward. The medical balloon assembly further includes a cutting element extending from the balloon and having a plurality of openings therein including a first opening and a second opening. The cutting element also includes a cavity extending from the first opening to the second opening. A portion of the outer wall extends into the cavity. A therapeutic agent is disposed in the cavity only in a region extending from the portion of the outer wall extending into the cavity to the first opening. The therapeutic agent at least partially fills the region. The balloon may be disposed about a catheter. Optionally, the flexible material forms a part of the balloon which is softer and/or thinner than the remainder of the balloon.

In at least one embodiment, the invention is directed to a method of treating a bodily vessel using a medical balloon assembly. The medical balloon assembly includes a balloon having an outer wall facing outward and an inner wall facing inward. The medical balloon assembly further includes a cutting element extending from the balloon and having a plurality of openings therein including a first opening and a second opening. The first opening is closed off by a portion of the outer wall. The cutting element further includes a passageway extending from the first opening to the second opening. The passageway is defined by the cutting element and the portion of the outer wall which closes off the passageway. Treatment agent is disposed in the passageway. The method includes positioning the medical balloon assembly in the vessel. The method further includes inflating the medical balloon assembly such that a portion of the outer wall extends into the passageway thereby causing at least some of the treatment agent to be delivered from the passageway to the vessel. The balloon may be disposed about a catheter.

### BRIEF DESCRIPTION OF THE FIGURES

The invention and the following detailed description of certain embodiments thereof may be understood with reference to the following figures:

FIG. 1 is a perspective view of a system having a catheter and a medical balloon operationally positioned in an upper body of a patient.

FIG. 2A is a view of a system with a catheter and a medical balloon assembly in a deflated state.

FIG. 2B is a view of the system of FIG. 2A in an inflated state.

FIG. 3A is a view of a system with a catheter and a medical balloon assembly in a deflated state.

FIG. 3B is a view of the system of FIG. 3A in an inflated state.

FIG. 4A is a cross-sectional view of a medical balloon assembly in a deflated state.

FIG. 4B is a cross-sectional view of the medical balloon assembly of FIG. 4A in an inflated state.

FIG. 5A is an enlarged cross-sectional view of a cutting element of a medical balloon assembly in a deflated state.

FIG. 5B is an enlarged cross-sectional view of a cutting element of a medical balloon assembly in an inflated state.

FIG. 6A is a cross-sectional view of a medical balloon assembly in a deflated state.

FIG. 6B is a cross-sectional view of the medical balloon assembly of FIG. 6A in an inflated state.

FIG. 7 is a cross-sectional view of a medical balloon assembly in another embodiment.

FIGS. 8A-8C illustrate cross-sectional views of a medical balloon assembly in another embodiment.

FIG. 9 is a cross-sectional view of a medical balloon assembly in another embodiment.

FIG. 10A is a schematic view of the system of FIG. 2A positioned at a treatment site, shown with the medical balloon assembly deflated.

FIG. 10B is a schematic view of the system of FIG. 2A positioned at the treatment site shown after inflation of the medical balloon assembly.

### DETAILED DESCRIPTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, and/or substituted for, elements depicted in another figure as desired.

The terms proximal and distal described in relation to various devices, apparatuses, and components as discussed in the subsequent text of the present invention are referred with a point of reference. The point of reference, as used in this description, is a perspective of an operator. The operator may be a surgeon, a physician, a nurse, a doctor, a technician, and the like who may perform the procedure of delivery and placement of the disclosed system/device into the patient's body as described in the present invention. The term proximal refers to an area or portion that is closer or closest to the operator during a placement procedure. The term distal refers to an area or portion that is further or farthest from the operator.

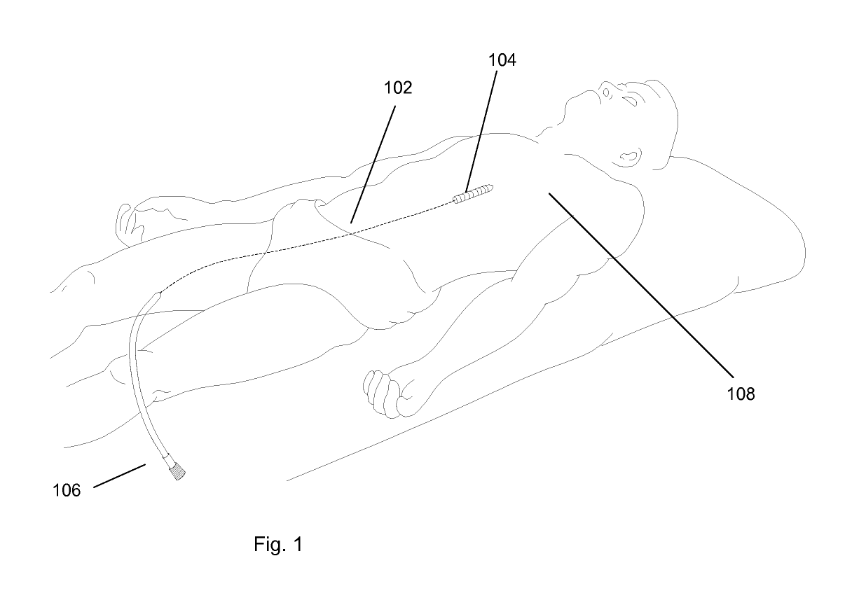

Referring now to FIG. 1, a medical system having a catheter 102, a medical balloon assembly 104, and a syringe 106 is shown for performing a medical procedure at an internal treatment site 108 of a patient. A pump or other suitable device may be used in place of a syringe. The medical system is shown positioned inside a patient's body to treat a lesion in an upper body artery of a human patient. Although the medical system is capable of performing a medical procedure in an upper body artery such as a coronary artery, those skilled in the pertinent art will quickly recognize that the use of the medical system as described herein is not limited to use in an artery, but, instead can be used in vascular conduits and other ductal systems such as a bile duct, a urinary track, and the like throughout the human body. Moreover, although FIG. 1 shows the system used in a human body, it is to be appreciated that the medical system can also be used in non-humans such as animals, if desired. Functionally, the medical system is configured to incise or cut a biological material within a body conduit. As used herein, the term “biological material” and its derivatives includes, but is not limited to, cellular matter including tissue (diseased, healthy or otherwise), deposits such as cholesterol and calcium deposits, and lesions which, for example, may consist of cellular matter and/or deposits.

FIG. 1 illustrates an exemplary embodiment of access to a bodily location. However, other approaches of access through different body locations other than those shown in FIG. 1, including, but not limited to, radial access via the arm, are within the scope of the present invention.

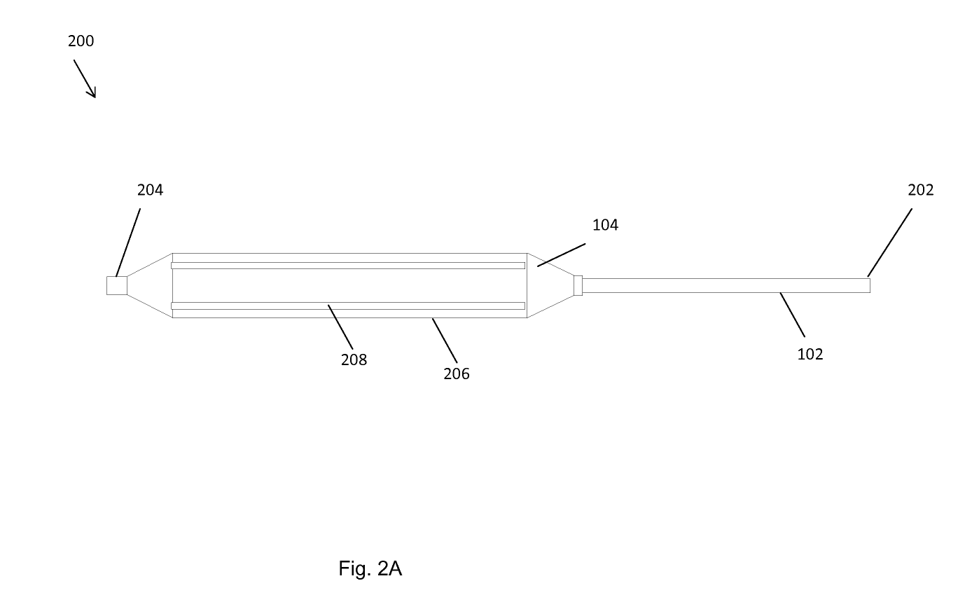

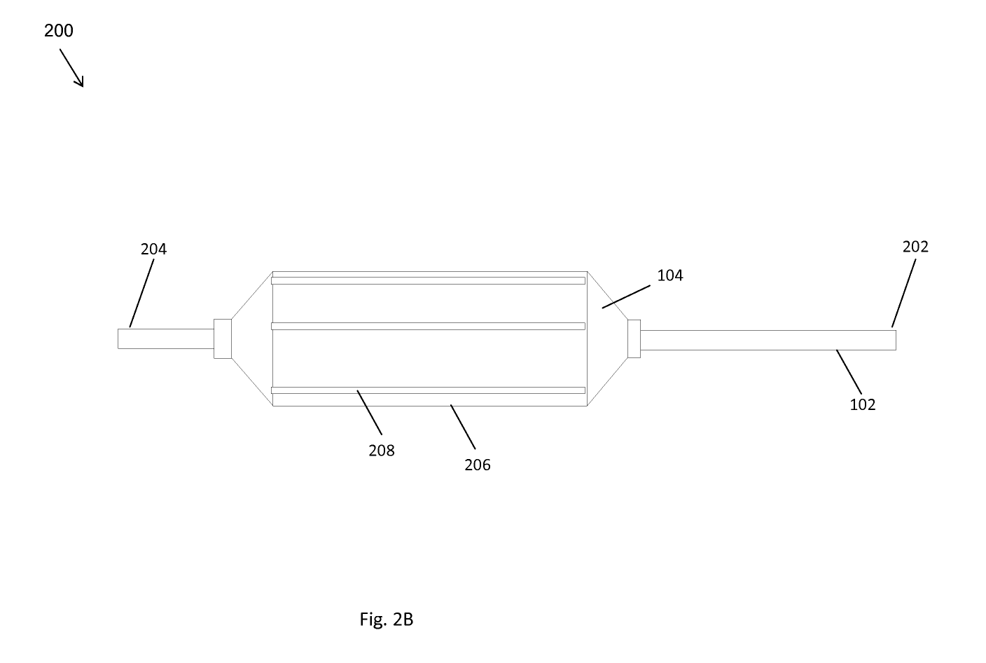

As indicated above, the present invention is embodied in a variety of forms. In at least one embodiment, an example of which is depicted in FIGS. 2A and 2B, the invention is directed to a system 200 with a proximal end portion 202 and a distal end portion 204, and having a catheter 102 and a medical balloon assembly 104 disposed about a catheter tube. The medical balloon assembly 104 is configured to expand from a first deflated configuration to a second inflated configuration. The deflated configuration of the medical balloon assembly 104 is illustrated in FIG. 2A and the inflated configuration is illustrated in FIG. 2B. The medical balloon assembly 104 includes an inflatable balloon 206 and one or more cutting elements 208.

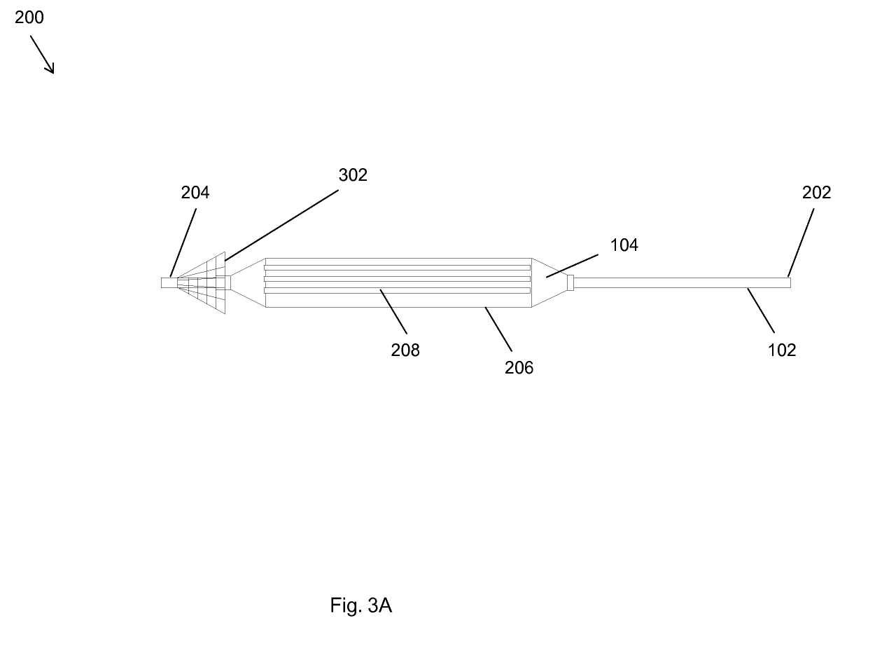

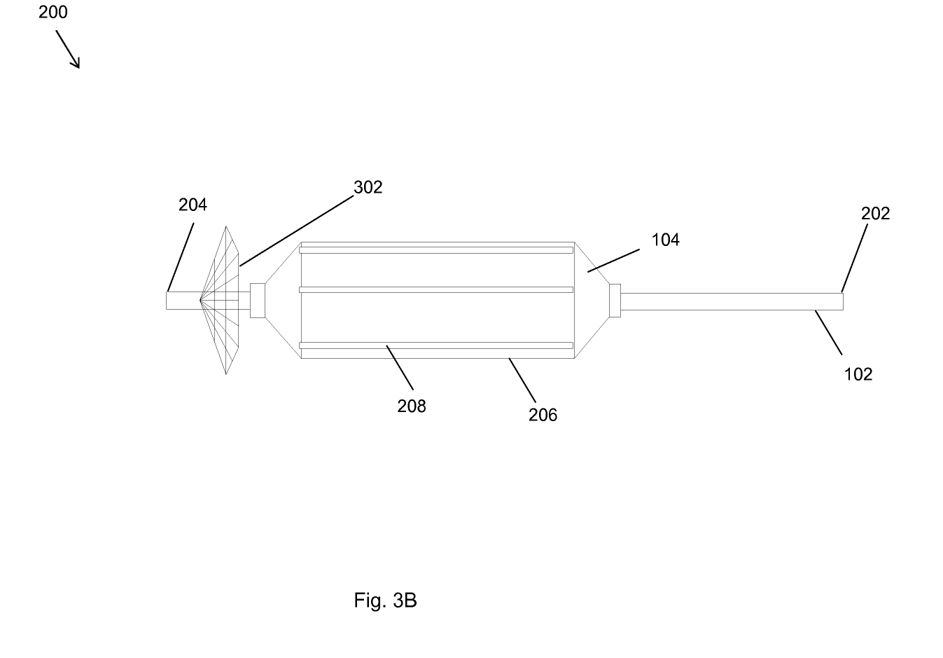

An illustration of an embodiment including a filter is shown in FIGS. 3A and 3B. In the embodiment shown, a filter 302 is coupled to the distal end portion 204 of the catheter tube. The filter 302 is configured to collect debris which may be caused by the action of the medical balloon assembly 104 and to protect the patient against embolic material. Although FIGS. 3A and 3B show the filter 302 coupled to the distal end portion 204 of the catheter tube, it is to be appreciated that the filter 302 can be coupled to the proximal end portion 202 or both the distal end portion 204 and proximal end portion 202. Further, as shown in FIGS. 3A and 3B, medical balloon assembly 104 is disposed about catheter 102. A filter such as the FilterWire EZ™ Embolic Protection System or any other suitable filter may optionally be used.

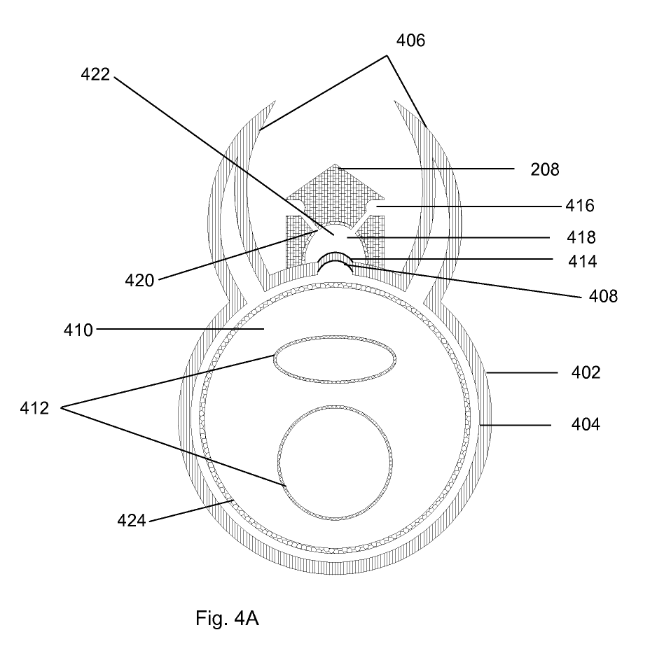

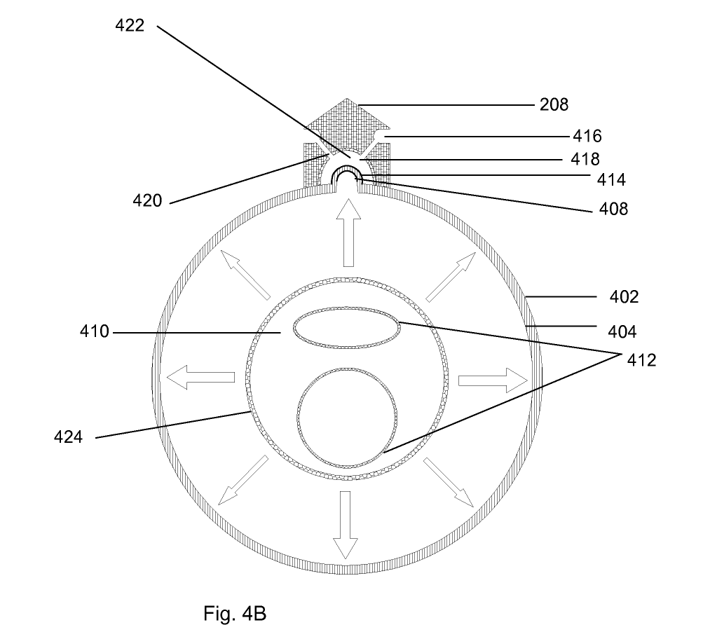

Referring now to FIGS. 4A and 4B, there is shown a cross-sectional view of a medical balloon assembly 104 having a balloon 206 with an outer wall 402 facing outward and an inner wall 404 facing inward. The balloon 206 is inflatable and configured to expand from a first deflated state illustrated in FIG. 4A to a second inflated state illustrated in FIG. 4B defined by a varying radial cross-section. The balloon 206 has at least one fold 406 (as illustrated in FIG. 4A) extending outward from the outer wall 402 of the balloon 206. Although FIG. 4A shows only two folds 406 of the balloon 206, it is within the scope of the invention for balloon 206 to include more than two folds or just one fold or no folds. The at least one fold 406 is visible only in the first deflated state. As the balloon is inflated, the fold typically disappears and the balloon assumes a circular cross-section, deviating only in the regions of the pinched portions extending into cutting elements 208.

Any suitable balloon fold may be used. Other examples of balloon folds include those described in U.S. Pat. No. 5,320,634, U.S. Pat. No. 7,494,612 and U.S. Pat. No. 7,972,351.

The outer wall 402 of the balloon 206 has at least a portion referred to as a pinched portion 408, extending radially outward relative to the rest of the outer wall 402. In this particular embodiment, the pinched portion 408 is made of a flexible material. Further, the outer wall 402 is optionally thinner along the pinched portion 408 than the rest of the outer wall 402 of the balloon 206. In another embodiment, the outer wall 402 is optionally softer along the pinched portion 408 than the rest of the outer wall 402 of the balloon 206. FIGS. 4A and 4B show only one pinched portion 408; it is to be appreciated that the balloon 206 can include more than one pinched portions.

An inner space 410 of the balloon 206 may include at least one lumen 412 configured to receive an inflation fluid adapted to pressurize the balloon 206 and make it expand from a first deflated state to a second inflated state as well as an optional lumen for receiving a guidewire. In the illustrated embodiments, two such lumens 412 are shown; however, it is to be appreciated that there could be only one or more than two lumens in the inner space 410. The at least one lumen may be disposed within a catheter tube 424.

The balloon 206 may be made of a polymeric material such as Tecothane®, PEBAX®), polyethylene, and the like or any other suitable balloon material including elastomeric materials, compliant materials and non-compliant materials.

The pinched portion of the balloon may be made of the same material as the remainder of the balloon. In such a case, the wall in the pinched region will typically be thinner than the remainder of the wall of the balloon. Optionally the wall in the pinched region may be softer than the remainder of the wall of the balloon. Where the pinched portion is made of a different material from that of the remainder of the balloon, the material of the pinched portion will be chosen so that it will bulge outward relative to the remainder of the balloon during inflation. To that end, the material in the pinched portion may be more flexible or less rigid as compared to the remainder of the balloon. Optionally, the pinched portion of the balloon may be made of a more compliant material than the non-pinch portion.

Alternatively, the balloon may be made by the process of co-extrusion of two polymers where one of them is a compliant material and the other is a non-compliant material. The non-compliant material may be removed by cold ablation in the regions of the pinched portion 408 using, for example, an excimer laser. Further the compliant material may be separated axially along the balloon.

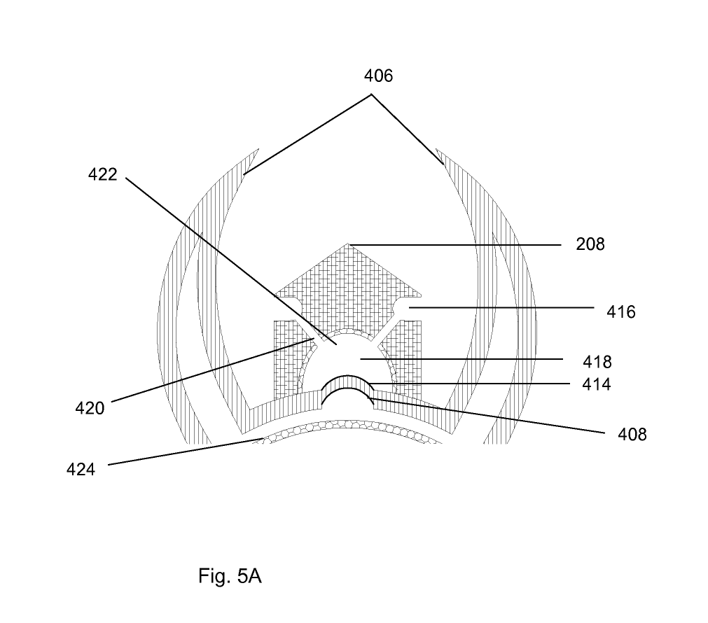

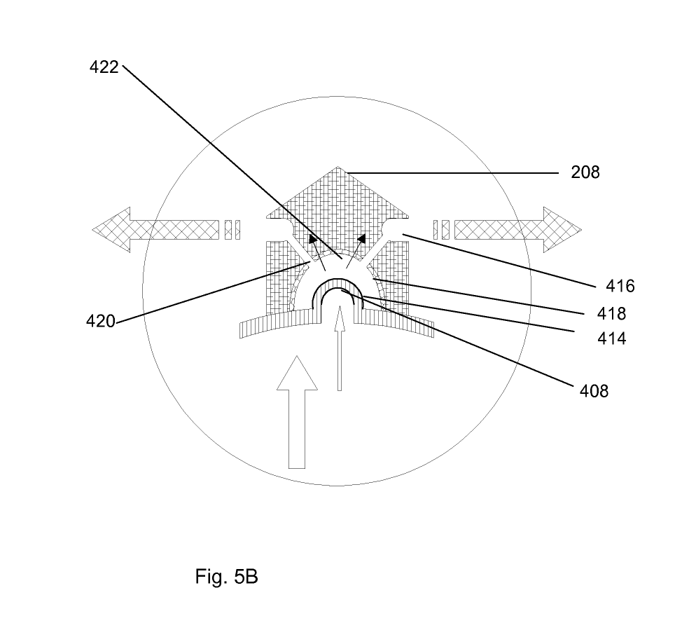

The embodiment shown in FIGS. 4A and 4B further includes a cutting element 208 extending from the balloon 206. An enlarged cross-sectional view of the cutting element 208 is shown in FIGS. 5A and 5B. FIG. 5A illustrates the deflated configuration and FIG. 5B illustrates the inflated configuration of the balloon. Cutting element 208 includes a plurality of openings therein, including a first opening 414 and a second opening 416, and a passageway 418 extending from the first opening 414 to the second opening 416. The first opening 414 is configured to be closed off by the portion of the outer wall 408 of the balloon 206 referred to as the pinched portion 408 above. The second opening 416 is provided in a side surface of the cutting element 208. In an embodiment, the passageway 418 is defined by the cutting element 208 and the pinched portion 408 of the outer wall 402 that closes off the passageway 418. The passageway 418 has a first volume in the first deflated state and the balloon 206 is constructed and arranged such that upon inflation of the balloon 206, the passageway 418 has a second volume that is less than the first volume. As shown, a set of two first openings 414 is provided in the cutting element 208 on substantially diametrically opposite locations; it is to be appreciated that there can be only one or more than two first openings 414.

In accordance with the invention, the cutting element 208 can be a cutting blade. The cutting blade is configured to cut the lesions/plaque inside a bodily vessel. The cutting element 208 may be made of suitable metals and metal alloys such as stainless steel, for example, 316L stainless steel, and cobalt-chromium alloys, for example L605 Co—Cr, high density polymers, or any other suitable materials. Typically, these cutting blades will be in the form of microsurgical blades, sometimes referred to as “atherotomes.” The cutting element can include any suitable mounting system for mounting the cutting blade on the balloon.

Desirably, as shown in FIGS. 4A-5B, a therapeutic agent 422 is disposed within the passageway 418. The therapeutic agent is deliverable from the passageway 418 upon an application of a radially outward force to the pinched portion 408 of the outer wall 402 which closes off the passageway 418. As the balloon is inflated, pinched portion 408 expands outward, thereby reducing the size of passageway 418 and forcing therapeutic agent therein out of the one or more openings 416.

It is within the scope of the invention for the cross-section of the passageway 418 to be uniform or non-uniform along its length as illustrated in FIGS. 4A-5B. In shown in FIGS. 4A-5B, another opening 420 may be provided in the cutting element 208 that is in fluid communication with the passageway 418. A portion of the passageway 418 below the additional opening 420 may be termed as a reservoir for therapeutic agent. The second and additional opening are on opposite sides of the cutting element. It is also within the scope of the invention for the second and any additional openings to be on the same side of the cutting element. It is further within the scope of the invention for the second and/or additional openings to run substantially the length of the cutting element or for a plurality of second and/or additional openings to run along the sides of the cutting element(s). The second and/or additional elements can also be positioned elsewhere along the cutting element(s). It is also within the scope of the invention to provide only one first opening and only one second opening and no additional openings in a cutting element as well as to provide one or more second openings and one or more additional openings in a cutting element. For example, the illustrated embodiment shows another additional opening 420 in fluid communication with the passageway 418.

It is within the scope of the invention for the pinched portion to extend into an opening in the cutting blade prior to inflation of the balloon. Upon inflation of the balloon, the pinched portion extends further into the opening. It is also within the scope of the invention for the pinched portion to not extend into the opening until the balloon is inflated.

In at least one embodiment of the invention, the cutting element may have a cavity therein defined by a space extending from the first opening 414 to the second opening 416. The volume of the cavity 418, prior to inflation, is the same as the volume after inflation. However, the pinched portion 408 extends across the first opening 414 after the inflation and into the cavity. In some embodiments, the pinched portion 408 is made of a flexible piece of material such that the first opening 414 is covered by the flexible piece of material that extends into the cavity 418 upon inflation of the balloon 206. In this embodiment, a therapeutic agent is disposed in the cavity 418. During inflation, the therapeutic agent is disposed in the cavity 418 only in a region extending from the portion of the outer wall 402 extending into the cavity 418 to the first opening 414. The therapeutic agent at least partially fills the region.

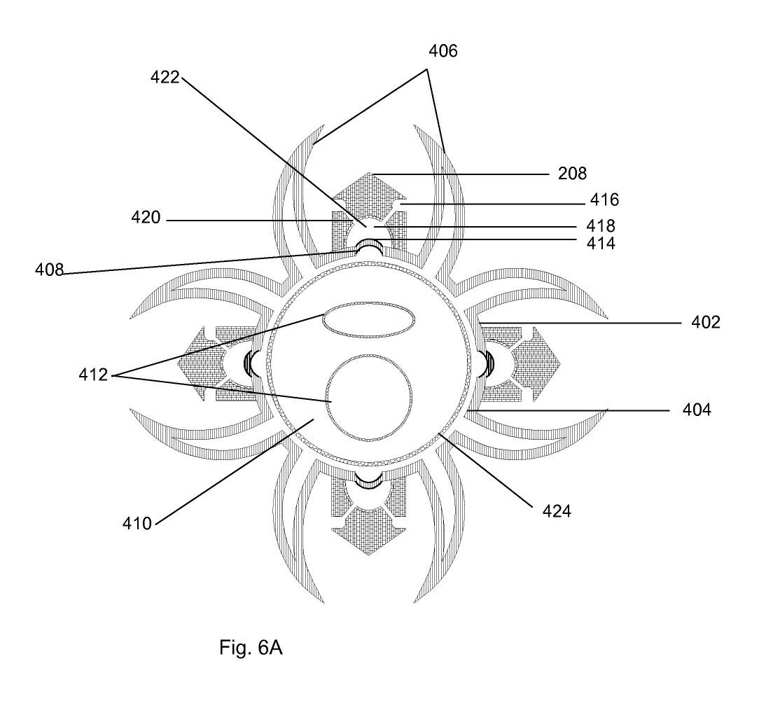

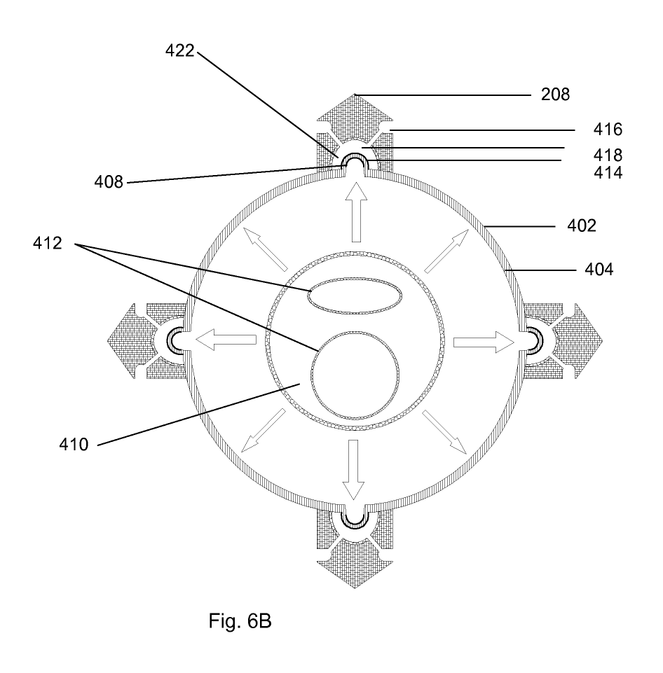

FIGS. 6A and 6B illustrate an inventive medical balloon assembly 104 having a plurality of cutting elements 208. FIG. 6A shows a cross-sectional view of the medical balloon assembly in the first deflated state. FIG. 6B shows a cross-sectional view of the medical balloon assembly 104 in the second inflated state. The medical balloon assembly includes a plurality of cutting elements such as the cutting element 208 described above. FIGS. 6A and 6B show four cutting elements 208; it is to be appreciated that the medical balloon assembly 104 can include more than four or less than four cutting elements 208. For example, there can be one, two, three or more cutting elements 208. Each of the cutting elements 208 includes a passageway or a cavity 418. Further, a pinched portion 408 is provided on the outer wall 402 of the balloon 206 for each of the passageways 418 that close off the first opening 414 in each of the cutting elements 208. The terms passageway and cavity 418 as defined with respect to the present invention have already been described above in conjunction with FIGS. 4A and 4B.

In this embodiment, a therapeutic agent is disposed in each of the passageways 418. The therapeutic agent is deliverable from the passageways 418 upon the application of a radially outward force to the balloon in general and, in particular, to the pinched portions 408 of the outer wall 402 which close off the passageways 418.

In general, the therapeutic agent for use in connection with the present invention can be any pharmaceutically acceptable therapeutic agent. Desirably, the therapeutic agent will be an agent that is approved by the United States Food and Drug Administration for use in humans or animals when incorporated in or on an implantable or insertable medical device. The invention also contemplates using therapeutic agents that have been approved by the regulatory agencies of countries other than the United States. The therapeutic agents can include an Active Pharmaceutical Ingredient (API). The API is formulated as one of a liquid form of an API matrix, a gel form of an API matrix, drug crystal, milled crystal, nano crystal, micro crystal, and polymorph. Desirable therapeutic agents include antiresteotic agents, anticoagulation agent, anti-inflammatory agents, analgesic agents, local anesthetic agents, antispasmodic agents, and combinations thereof.

Antirestenotic agents such as those currently used for drug eluting vascular products include paciltaxel, everolimus, sirolimus (rapamycin), zotarolimus, biolimus A9, as well as others. Anticoagulation agents include heparin, warfarin, and the like. Analgesic agents include narcotic and non-narcoticanalgesics. Narcotic analgesic agents include alfentanil, allylprodine, alphaprodine, anileridine, benzylmorphine, bezitramide, buprenorphine, and the like. Non-narcotic analgesics include aceclofenac, acetaminophen, acetaminosalol, acetanilide, acetylsalicylsalicylic acid, alclofenac, and the like.

Anti-inflammatory agents include steroidal and non-steroidal anti-inflammatory agents. Examples of nonsteroidal anti-inflammatory drugs include aminoarylcarboxylic acid derivatives such as enfenamic acid, etofenamate, flufenamic acid, and the like. Examples of steroidal anti-inflammatory agents (glucocorticoids) include 21-acetoxyprefnenolone, aalclometasone, algestone, amicinonide, beclomethasone, betamethasone, budesonide, chloroprednisone, and the like. Antispasmodic agents include alibendol, ambucetamide, aminopromazine, apoatropine, bevonium methyl, and the like.

Antispasmodic agents include alibendol, ambucetamide, aminopromazine, apoatropine, bevonium methyl sulfate, bietamiverine, butaverine, butropium bromide, and the like.

Once disposed within the body of the patient, the therapeutic agent is released from the medical balloon assembly 104. The dose rate may vary depending on the application and the size of the patient.

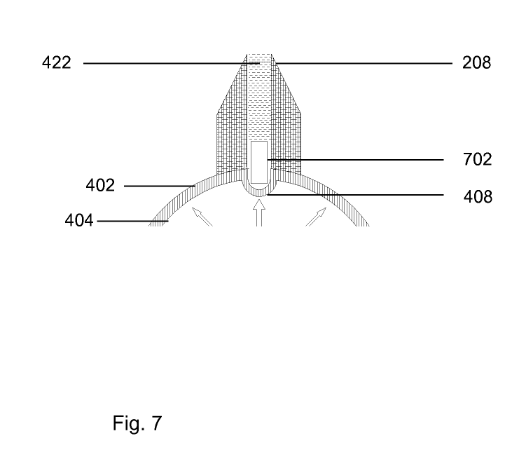

As illustrated in FIG. 7, another embodiment including other ways of eluting the drug through the pinched portion 408 is now described. Optionally, the cutting element 208 as discussed above may include an integral plunger 702. As the balloon is inflated, the pinched portion 408 expands outward which pushes the integral plunger 702 upward, thereby forcing the therapeutic agent or medication 422 out.

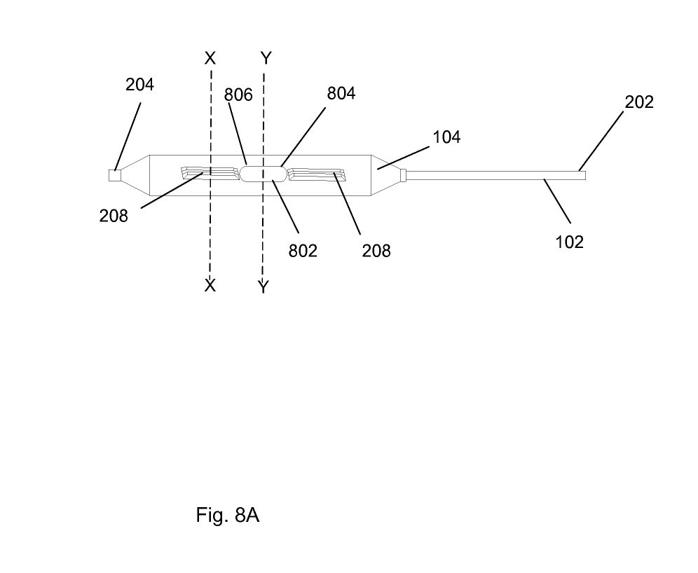

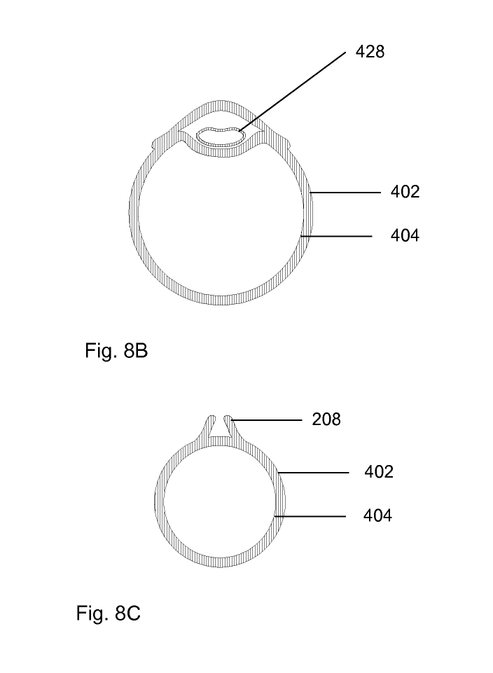

In still some other embodiments, as illustrated in FIG. 8A, the medical balloon assembly 104 further includes a pump portion 802 having a proximal end portion 804 and a distal end portion 806, a lumen/channel which is an integral part of the pump portion 802 and the cutting element 208. In one embodiment, the pump portion 802 is disposed in the middle of the balloon (as shown in FIG. 8A). In another embodiment, the pump portion 802 is disposed at the proximal end portion of the balloon or the distal end portion of the balloon. In another embodiment, the pump portion 802 is disposed at both ends of the balloon. In one embodiment, the cutting element 208 is attached and extends from the proximal end portion 804 and the distal end portion 806 of the pump portion 802. In another embodiment, the cutting element 208 is an integral part of the pump portion 802.

As the balloon is inflated, the pump portion 802 may be compressed by the vessel walls. As the pump is compressed, the therapeutic agent disposed inside the pump portion 802 is forced out of the pump, desirably via a lumen/channel which extends into or is in fluid communication with the cutting elements 208 and, in turn, forced out of the cutting elements at a treatment site. The channel as discussed above may be an integral part of the pump portion 802. The channel could, optionally, begin at either end of the pump portion 802 (the proximal end portion 804 or distal end portion 806) or alternatively at both ends of the pump portion 802.

In another embodiment, the therapeutic agent may already be present in the lumen/channel 412 and the cutting element 208. As the balloon is inflated and the pump portion 802 compressed by the vessel walls, the therapeutic agent is forced out. FIG. 8B illustrates a cross sectional view of a portion from FIG. 8A taken along Y-Y axis and FIG. 8C illustrates a cross sectional view of a portion from FIG. 8A taken along X-X axis.

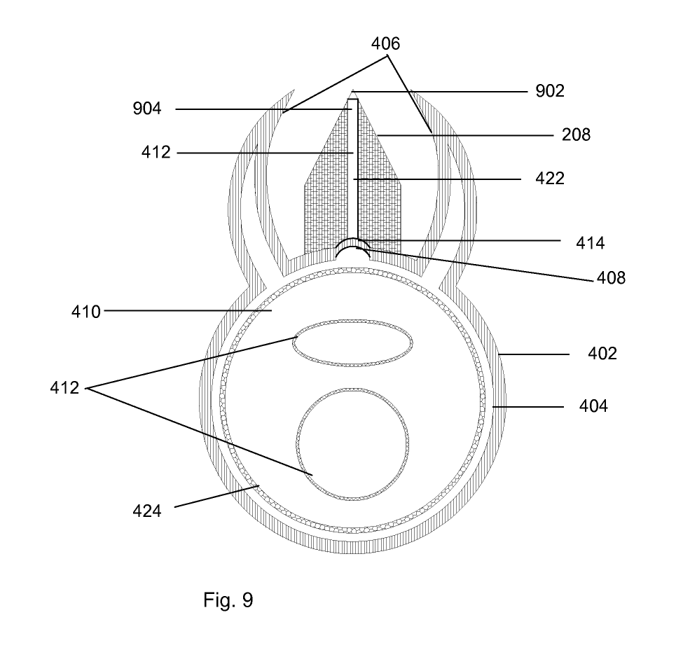

In some embodiments, the medical balloon assembly 104 may include a cutting element 208 that may include a brittle polymeric material which plugs opening 904 and prevents the therapeutic agent 422 from being forced out. FIG. 9 illustrates the medical balloon assembly 104 with a brittle tip 902. The brittle tip 902 is configured to break when subjected to an adequate shearing force. As the balloon expands and rotates, the brittle tip 902 breaks and the therapeutic agent 422 is forced out of the broken brittle tip 902 and contacts the tissue at almost the same time as the cutting element 208 touches the lesion for cutting.

The invention is also directed to a medical device comprising a catheter and a self-expanding stent. The medical device further comprises a sleeve disposed about the self-expanding stent. The sleeve comprises one or more cutting elements such as those disclosed herein. The stent and sleeve are covered by a removable sheath. The sheath may be made of Teflon, Pebax®, Dacron® or other suitable materials including graft materials. The sheath may be braided or woven or of any other suitable configuration. The sheath may be withdrawn by pulling on it, either directly or via a pull wire. U.S. Pat. No. 6,120,522 discloses an example of a delivery system for a self-expanding stent which may be modified by the inclusion of a sleeve such as that disclosed herein.

When the sheath is withdrawn from over the stent and sleeve, the stent may self-expand. The self-expanding stent forces the sleeve against the vessel wall resulting in the delivery of therapeutic agent. The sleeve may have pinched portions to effect the delivery of the therapeutic agent or may have one or more pump portions.

In one embodiment, the sleeve may remain in place after delivery. In othero embodiments, the sleeve may be removed.

Optionally, the sleeve including the cutting element(s) may be made of a bioabsorbable material such as PLA, PLGA, or any other suitable polymer including polymers which degrade by swelling and hydrolysis, polyester amides which are subject to enzymatic degradation and the like.

Illustrations of some possible embodiments where the cutting element 208 is engaged to the medical balloon assembly 104 are shown in FIGS. 3A-6B. The cutting element 208 can be a separate part attached to a portion of the balloon 206 or it can be an integrated component of the medical balloon assembly 104. This invention encompasses embodiments where any number of cutting elements 208 may be a part of, may be engaged to, or may protrude from the balloon 206. In at least one possible embodiment, for example, at least four cutting elements 208 are radially affixed or otherwise engaged to the balloon 206. The radially positioned cutting elements 208 can be equidistant to each other relative to the circumferential cross section of the outer wall 402 of the balloon 206 or they can be positioned at unequal intervals relative to the circumferential cross section of the outer wall 402 of the balloon 206.

In one embodiment, the cutting element 208 can both self deploy and retract down on to the surface of the outer wall 402 of the balloon 206 as needed through the expansion of the balloon 206 or through an independent expansion or retraction mechanism.

In embodiments having multiple cutting elements 208, the cutting elements 208 may be positioned in a uniform or non-uniform distribution of the balloon's outer wall 402. In some possible embodiments, different kinds of cutting elements 208 (including but not limited to) cutting blades known in the art, and/or those illustrated in conjunction with various figures may be affixed to, may protrude from, or may be otherwise engaged to the balloon 206. In addition, the cutting elements 208 need not be at uniform distance from the surface of the balloon 206 and differing portions of the cutting element 208 can be closer or farther away from the surface of the balloon 206. The cutting elements 208 can taper downwards towards the terminal end of the cutting element or downwards away from the terminal end. The terminal end of the cutting element corresponds to the portion of the cutting element which is furthest from the balloon surface. The cutting elements 208 can also be serrated.

In accordance with the illustrated embodiments, some specific types of therapeutic agent delivery mechanisms are described, it is to be appreciated that several other types of mechanisms and designs may be employed without limiting the scope of the present invention. For example, other types of hollow sections may be provided in the cutting element to store and deliver the therapeutic agent to the treatment site 108.

The types of medical devices capable of supporting mounted cutting elements 208 include, but are not limited to, stents, sheaths, grafts, shafts, catheters, balloons or any combination thereof

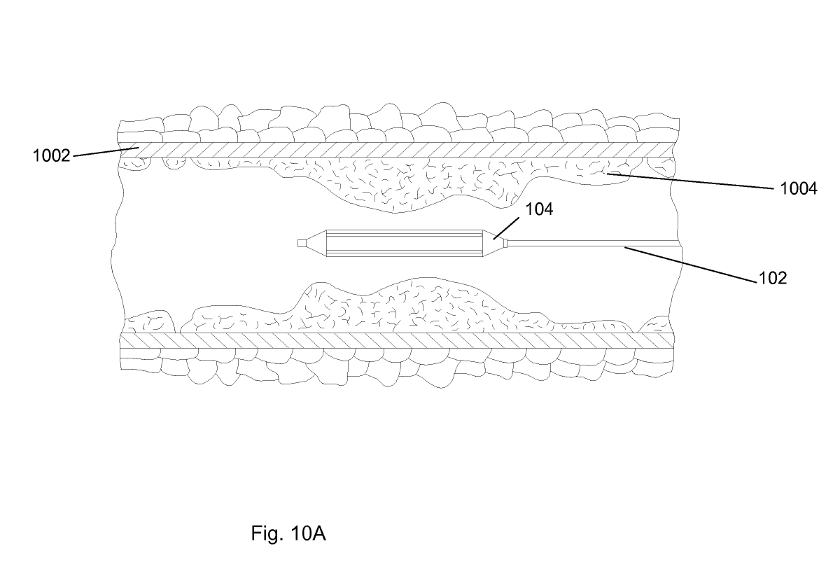

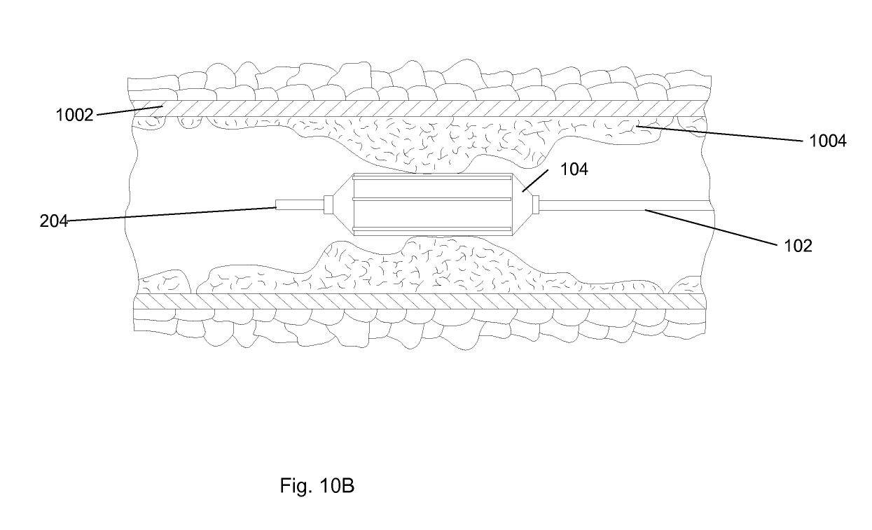

In a typical use, the distal end 204 of the catheter 102 supporting the medical balloon assembly 104 is inserted into a vasculature of the patient using a peripheral artery, such as the femoral artery, for access. The catheter 102 along with the medical balloon assembly 104 is then advanced until the distal end 204 of the catheter 102 is positioned slightly past a treatment site where an initial cutting operation is to be performed. FIGS. 10A-10B illustrate positioning of the catheter 102 and the medical balloon assembly 104 within a coronary artery 1002 that is constricted by a lesion 1004.

Once the medical balloon assembly 104 is properly positioned at the treatment site, a syringe 106, pump or other suitable device coupled to a proximal end 202 of the catheter 102 is activated to pass a fluid through an inflation lumen 412 and into the inflatable balloon 206 being inflatable from a first deflated configuration to a second inflated configuration. As the inflatable balloon 206 is pressurized radially by the flow of the fluid, the pinched portion 408 of the outer wall 402 of the balloon 206 is pushed outward, thereby causing some of the treatment agent to be delivered from the passageway/cavity 418 to the treatment site. Simultaneously, the cutting element is applied to a wall of the vessel in the vicinity of the lesion 1004 such that the cutting element notches or cuts down a portion of the lesion 1004. Therefore, the cutting and therapeutic agent eluting operations occur simultaneously.

In accordance with at least some embodiments, the medical balloon assembly 104 of the disclosed invention offers effective lesion cutting and localized treatment of the treatment site for use in a minimally invasive peripheral and/or cardiovascular interventional environment. The disclosed medical balloon assembly 104 involves utilization of cutting mechanisms that are configured to selectively score/cut the target vessel lesions and then release a suitable therapeutic agent (such as an API) to the prescored site in a controlled manner. This helps in improving the vascular interaction and subsequent availability of the therapeutic agent while negating the effects of agent wash-off due to the normal environmental pulsatile flow as experienced in the native vasculature. The medical balloon assembly 104 is advanced into the vessels to a treatment site which is typically an area that has evidence of slight or advanced atherosclerotic related disease.

In accordance with at least some embodiments, the medical balloon assembly 104 is integratable with various balloon designs such as the Flextome® brand cutting balloon dilatation device manufactured by the Boston Scientific or other atherotomy cutting balloons. Other examples of cutting balloons that can be used in conjunction with this invention are disclosed in US publication 2009/0192537 and U.S. Pat. Nos. 5,320,634, 6,632,231, 7,172,609, and 7,070,576. In addition, the medical balloon assembly 104 is integratable with various therapeutic agent formulation technologies used for Drug Eluting Stents or Drug Eluting Balloons.

In accordance with at least some embodiments, the medical balloon assembly 104 provides dual purposes with a single design. The disclosed medical balloon assembly 104 is configured to eliminate the need for two separate devices for lesion cutting and subsequent therapeutic agent delivery.

In accordance with at least some embodiments, the medical balloon assembly 104 is configured to be used in applications in situations where total chronic vasculature occlusion is diagnosed. Further, in accordance with at least some embodiments, the medical balloon assembly 104 is also configured to be used in applications in situations where partial chronic vasculature occlusion is diagnosed.

In accordance with at least some embodiments, the balloon material of the medical balloon assembly 104 may be manufactured by extrusion, molding or any other suitable balloon manufacturing technique. The balloons may be manufactured to have a single layer of material or multiple layers of material. Typically, the balloon will be manufactured to have one or more pinched portions

Further, the cutting element 208 may be manufactured from steel or other suitable hardened material as discussed herein. Also, an appropriate size passageway 418 and opening in the cutting element 208 is then provided in the cutting element 208. The passageway and openings may be etched, drilled, molded, or laser cut into the cutting element. Any other suitable manufacturing technique may also be used to provide the passageway(s) or cavity(ies) in the cutting element.

The cutting elements may then be fitted onto the balloon pinched portions in such a way so as to allow the balloon to expand into the passageway or cavity in the balloon upon inflation of the balloon.

Typically, therapeutic agent will be provided in the passageway or cavity prior to fitting the cutting element onto the pinched portion.

The invention is also directed, in or more embodiments, to an expandable medical device having a cavity therein comprising therapeutic agent. The cavity has at least one opening configured to allow for delivery of the therapeutic agent therefrom to a region outside of the medical device. The medical device is configured such that on expansion of the medical device, a portion of the device extends into the cavity thereby causing some of the therapeutic agent to be expelled from the cavity. Typically, the medical device will include an inflatable balloon, a portion of which expands outward in the cavity to expel the therapeutic agent. Also typically, the cavity will be provided in a cutting blade which forms a portion of the medical device. In the case of a balloon, a portion of the balloon may, optionally, extend into the cavity prior to inflation.

A description of some exemplary embodiments of the invention is contained in the following numbered paragraphs:

1. A medical device comprising a balloon assembly, the balloon assembly comprising:

a balloon having an outer wall and an inner wall, the outer wall facing outward and the inner wall facing inward; and

a cutting element extending from the balloon and having an opening therein leading into a recess in the cutting element, a portion of the balloon extending into the recess, the recess having a therapeutic agent disposed therein.

2. The medical device of statement 1 wherein the balloon is not inflated.

3. The medical device of statement 1 wherein the balloon is inflated.

4. The medical device of any of statement 1-3 comprising a plurality of said cutting elements.

5. The medical device of any of statements 1, 2 and 4 wherein the portion of the balloon extending into the recess is configured to extend further into the recess upon inflation of the balloon so as to expel at least some of the therapeutic agent from the recess.

6. The medical device of any of statements 1, 2, 4 and 5 wherein the recess is in the form of a channel, the balloon configured to extend further into the recess upon inflation of the balloon so as to expel at least some of the therapeutic agent from the recess.

7. The medical device of any of statements 1-6 wherein the balloon includes at least one fold, the fold extending alongside the cutting element.

8. The medical device of any of statements 1-7 wherein the balloon includes a plurality of folds which extend outward at least partially in a radial direction of the balloon, the cutting element disposed between two of the folds.

9. A medical device comprising a balloon assembly, the balloon assembly comprising:

a balloon having an outer wall and an inner wall, the outer wall facing outward and the inner wall facing inward; and

a cutting element extending from the balloon and having an opening therein leading into a recess in the cutting element, the recess having a therapeutic agent disposed therein, a portion of the balloon configured to extend into the recess upon expansion of the balloon so as to expel at least some of the therapeutic agent from the recess.

10. The medical device of statement 9 comprising a plurality of said cutting elements.

11. The medical device of any of statements 9 and 10 wherein the recess is in the form of a channel.

12. The medical device of any of statements 9-11 wherein the balloon includes at least one fold, the fold extending alongside the cutting element.

13. The medical device of any of statements 9-12 wherein the balloon includes a plurality of folds which extend outward at least partially in a radial direction of the balloon, the cutting element disposed between two of the folds.

14. The medical device of any of statements 1-13 wherein the opening is in a side of the cutting element.

15. The medical device of any of statements 1-14 wherein the portion of the balloon which extends into the recess or which is configured to be extendable into the recess is thinner or softer or made of a more flexible material than the remainder of the balloon.

16. A medical device comprising a balloon assembly, the balloon assembly comprising:

a balloon having an outer wall and an inner wall, the outer wall facing outward and the inner wall facing inward; and

a cutting element extending from the balloon and having a plurality of openings therein including a first opening and a second opening, a cavity extending from the first opening to the second opening, a portion of the outer wall extending into the cavity;

wherein a therapeutic agent is disposed in the cavity only in a region of the cavity extending from the portion of the outer wall extending into the cavity to the first opening, the therapeutic agent at least partially filling said region.

17. A method of treating a bodily vessel comprising the step of:

positioning a medical balloon assembly in the bodily vessel, the medical balloon assembly comprising
a balloon having an outer wall and an inner wall, the outer wall facing outward and the inner wall facing inward;
a cutting element extending from the balloon and having a plurality of openings therein including a first opening and a second opening, a passageway extending from the first opening to the second opening, the first opening closed off by a portion of the outer wall, wherein the passageway is defined by the cutting element and the portion of the outer wall which closes off the passageway; and
treatment agent disposed in the passageway;

inflating the medical balloon assembly such that the portion of the outer wall extends into the passageway thereby causing at least some of the treatment agent to be delivered from the passageway to the vessel.

18. The method of statement 17, wherein the cutting element is in the form of a blade and further comprising the step of applying the cutting element to a wall of the vessel in the vicinity of a legion.

The above disclosure is intended to be illustrative and not exhaustive. This description will suggest many variations and alternatives to a person of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

What is claimed is:

1. A medical device comprising a balloon assembly, the balloon assembly comprising:
a balloon having an outer wall and an inner wall, the outer wall facing outward and the inner wall facing inward; and
a cutting element extending from the balloon and having a plurality of openings therein including a first opening and a second opening, a passageway extending from the first opening to the second opening, the first opening closed off by a portion of the outer wall, wherein the passageway is defined by the cutting element and the portion of the outer wall which closes off the passageway.

2. The medical device of claim 1, the passageway having a first volume prior to expansion of the balloon, wherein the balloon is constructed and arranged such that upon inflation of the balloon, the passageway has a second volume which is less than the first volume.

3. The medical device of claim 1 further comprising a therapeutic agent disposed within said passageway.

4. The medical device of claim 1 further comprising a plurality of said cutting elements, each of the cutting elements including a passageway therein.

5. The medical device of claim 4 wherein a therapeutic agent is disposed in each of said passageways.

6. The medical device of claim 1 further comprising a therapeutic agent disposed within said passageway, the therapeutic agent being deliverable from the passageway upon the application of a radially outward force to the portion of the outer wall which closes off the passageway.

7. The medical device of claim 1 wherein the second opening is in a side surface of the cutting element.

8. The medical device of claim 1 wherein the inflatable balloon comprises at least one fold which faces the second opening.

9. The medical device of claim 1 wherein the passageway has therapeutic agent disposed therein and the cutting element has an additional opening therein in fluid communication with the passageway, the opening configured so as to allow displacement of at least some of the therapeutic agent from the passageway upon expansion of the balloon.

10. The medical device of claim 1 wherein the portion of the outer wall which closes off the passageway is thinner than rest portion of the outer wall.

11. The medical device of claim 1 wherein the portion of the outer wall which closes off the passageway is softer than the remainder of the outer wall.

12. A medical device comprising a balloon assembly, the balloon assembly comprising:
an inflatable balloon having an outer wall and an inner wall, the outer wall facing outward and the inner wall facing inward; and
a cutting element extending from the balloon and having a plurality of openings therein including a first opening and a second opening, a cavity extending from the first opening to the second opening, a portion of the outer wall extending across the first opening,
wherein the outer wall is constructed and arranged such that in an inflated state of the balloon, a portion of the outer wall extends into the cavity.

13. The medical device of claim 11 further comprising a plurality of said cutting elements.

14. The medical device of claim 11 further comprising a therapeutic agent disposed within a portion of said cavity, the balloon configured such in the inflated state at least some of the therapeutic agent is displaced from said cavity.

15. A medical device comprising a balloon assembly, the balloon assembly comprising:
an inflatable balloon having a balloon wall; and
a cutting element extending from the balloon and having a plurality of openings therein including a first opening and a second opening, a cavity extending from the first opening to the second opening,
wherein the first opening is covered by a flexible piece of material which is configured to extend into the cavity upon inflation of the balloon.

16. The medical device of claim 14 further comprising a therapeutic agent disposed within a portion of said cavity.

17. The medical device of claim 15, the material extending into the cavity prior to inflation of the balloon, wherein upon inflation of the balloon, the material extends further into the cavity than prior to inflation of the balloon.

18. The medical device of claim 15 further comprising a plurality of said cutting elements.

19. The medical device of claim 18 wherein each of the cutting elements has therapeutic agent disposed therein.

20. The medical device of claim 15 wherein the flexible material forms a part of the balloon which is softer and/or thinner than the remainder of the balloon.
